(19) United States
(12) Patent Application Publication
(10) Pub. No.: US 2024/0408356 A1
(43) Pub. Date: Dec. 12, 2024

(54) CATHETER HANDLE CONTROLLING MULTIPLE DEGREES OF FREEDOM OF A SET OF NESTED CATHETERS

(71) Applicant: CARDIAC SUCCESS LTD., Yokneam (IL)

(72) Inventors: David NEUSTADTER, Nof Ayalon (IL); Boaz MANASH, Zichron Yaakov (IL)

(21) Appl. No.: 18/702,122

(22) PCT Filed: Oct. 25, 2022
(86) PCT No.: PCT/IB2022/060251

§ 371 Date: Apr. 17, 2024

Related U.S. Application Data
(60) Provisional application No. 63/271,713, filed on Oct. 26, 2021.

(51) Int. Cl. A61M 25/01; A61M 25/00

(52) U.S. Cl. CPC A61M 25/0136; A61M 25/0147; A61M 2025/0004

(57) ABSTRACT

Embodiments of the present disclosure include a cardiac device, having: a set of two nested catheters including an outer catheter and an inner catheter; and a single handle associated with the set of two nested catheters, including an outer shell and an inner catheter mount assembly configured to slide longitudinally within the outer shell. The inner catheter mount assembly includes a rotatable knob configured to be rotated from an exterior of the handle. The inner catheter mount assembly includes a deflection actuator configured to be actuated from an exterior of the handle. The inner catheter's distal end is distal to the distal end of the outer catheter, and a total range of translation of the inner catheter relative to the outer catheter is greater than one third of an overall length of the handle.

### CROSS-REFERENCE TO RELATED APPLICATIONS

This application claims priority from U.S. Provisional Patent Application No. 63/271,713, filed Oct. 26, 2021, which is fully incorporated herein by reference.

### TECHNICAL FIELD

The embodiments of the present disclosure relate in general to devices and methods involving a set of multiple nested catheters connected to a single handle. More specifically, the embodiments of the present disclosure relate to a single handle and methods to control multiple degrees of freedom of movement of the multiple nested catheters.

### BACKGROUND

In some transcatheter procedures, particularly structural heart procedures in which catheters often need to maneuver within the chambers of the heart, there is a need to control complex movements involving multiple degrees of freedom of the catheters. Often a single catheter is not sufficient to provide the flexibility and degrees of freedom necessary, and a pair or set of nested catheters is required, each catheter providing additional degrees of freedom. For instance, an outer catheter might provide deflection in one plane and an inner catheter might provide deflection in a different plane that is adjustable relative to the plane of the deflection of the outer catheter. An outer catheter might provide deflection at one location and an inner catheter might provide additional deflection at another location that is adjustable relative to the location of the deflection of the outer catheter. Many additional combinations of degrees of freedom exist when using multiple nested catheters.

One of the challenges in designing and using multiple nested catheters to provide multiple degrees of freedom is the control of all of these degrees of freedom. Generally, each catheter has a handle at its proximal end that contains actuators for each degree of freedom. In/out movement and rotation of the catheter are generally performed by simply pushing the handle in or out and by rotating the handle, and deflection of a deflectable catheter is generally controlled by moving an actuator on the handle. When multiple nested catheters are being used together to manipulate the tip of the innermost catheter, all degrees of freedom of all of the catheters must be controlled simultaneously, which can be quite challenging. One solution for this challenge is to have multiple operators working together to control the multiple catheters, however, synchronization and coordination between multiple operators can be quite challenging. Other solutions include a stand which holds the handles of the catheters or an adapter that connects that handles to each other in a semi-rigid manner. These solutions allow some degrees of freedom to be held firmly while allowing others to be controlled and manipulated. This allows a single operator to control multiple handles, however, these solutions are cumbersome and do not allow convenient and rapid switching between degrees of freedom, let alone simultaneous control of all degrees of freedom.

Accordingly, there is a need for an improved handle design that provides more convenient control of the multiple degrees of freedom provided by a device containing multiple nested catheters.

### SUMMARY

Presently disclosed embodiments recognize that a need exists for an improved handle design that provides more convenient control of the multiple degrees of freedom for a set of nested catheters. The embodiments of the present disclosure include devices and methods of providing a set of multiple nested catheters connected to a single handle. Advantageously, the exemplary embodiments provide devices and methods of providing a single handle that is configured to control multiple degrees of freedom of movement of the multiple nested catheters. Various embodiments of the disclosure may include one or more of the following aspects.

Consistent with an embodiment of the present disclosure, a cardiac device is provided. The cardiac device may include a set of at least two nested catheters including at least an outer catheter and an inner catheter. The cardiac device may include a single handle associated with the set of at least two nested catheters, the single handle including an outer shell and an inner catheter mount assembly configured to slide longitudinally within the outer shell. The outer catheter may be associated with the outer shell of the handle, such that translation of the outer shell is configured to translate the outer catheter and the inner catheter together, and rotation of the outer shell is configured to rotate the outer catheter and the inner catheter together. The inner catheter may be associated with the inner catheter mount assembly, such that sliding of the inner catheter mount assembly within the outer shell of the handle is configured to induce translation of the inner catheter relative to the outer catheter. The inner catheter mount assembly may include a rotatable knob configured to be rotated from an exterior of the handle, such that rotation of the rotatable knob is configured to induce rotation of the inner catheter relative to the outer catheter. The inner catheter mount assembly may include a deflection actuator configured to be actuated from an exterior of the handle, such that actuation of the deflection actuator is configured to induce deflection of a portion of the inner catheter that extends beyond a distal end of the outer catheter. A range of translational motion of the inner catheter relative to the outer catheter may include: the inner catheter being positioned such that its distal end is distal to the distal end of the outer catheter; and a total range of translation of the inner catheter relative to the outer catheter being greater than one third of an overall length of the handle.

Some embodiments may include a set of multiple nested catheters connected to a single handle that is configured to control multiple degrees of freedom of movement of the multiple nested catheters. Optionally, the degrees of freedom of movement controlled by the handle may include translational movement of each catheter, rotation of each catheter, and deflection of each catheter in one or more planes.

In some embodiments, the outer catheter may be associated with the outer shell of the handle and may be configured such that translation of the outer shell of the handle induces translation of the outer catheter and rotation of the outer shell of the handle induces rotation of the outer catheter. In some embodiments the outer catheter may be deflectable, and the deflection of the outer catheter may be controlled by an actuator associated with the outer shell of the handle.

In some embodiments, an inner catheter that is located inside a lumen of the outer catheter may be associated with an inner catheter mount which is located inside the outer shell of the handle and which can rotate and/or translate relative to the outer shell of the handle. In some embodiments, the inner catheter and inner catheter mount may be configured such that rotation and translation of the inner catheter mount induce rotation and translation of the inner catheter.

Additional objects and advantages of the embodiments will be set forth in part in the description that follows, and in part will be obvious from the description or may be learned by practice of the embodiments. The objects and advantages of the embodiments will be realized and attained by means of the elements and combinations particularly pointed out in the appended claims.

It is to be understood that both the foregoing general description and the following detailed description are exemplary and explanatory only and are not restrictive of the claims.

### BRIEF DESCRIPTION OF THE DRAWINGS

Some features of disclosed embodiments are set forth with particularity in the claims that follow. Additional details of the features and advantages of the disclosed embodiments will be obtained by reference to the following detailed description that sets forth illustrative examples, in which the disclosed principles are utilized, and the accompanying drawings of which:

FIG. 1A illustrates an exemplary cardiac device, consistent with an embodiment of the present disclosure;

FIG. 1B illustrates a translational movement of an exemplary cardiac device, consistent with an embodiment of the present disclosure;

FIG. 1C illustrates a rotational movement of an exemplary cardiac device, consistent with an embodiment of the present disclosure;

FIG. 1D illustrates a deflection of an exemplary cardiac device, consistent with an embodiment of the present disclosure;

FIG. 2 illustrates an exemplary deflection actuator of a cardiac device, consistent with an embodiment of the present disclosure;

FIG. 3 illustrates the components of an exemplary deflection actuator of a cardiac device, consistent with an embodiment of the present disclosure;

FIG. 4 illustrates an exemplary rotatable knob, consistent with an embodiment of the present disclosure;

FIG. 5 illustrates the components of an exemplary rotatable knob, consistent with an embodiment of the present disclosure;

FIG. 6A illustrates the components of an exemplary inner catheter mount assembly, consistent with an embodiment of the present disclosure;

FIG. 6B illustrates an exemplary inner carriage configuration, consistent with an embodiment of the present disclosure.

FIG. 7A illustrates one example of a winch, consistent with an embodiment of the present disclosure;

FIG. 7B illustrates another example of a winch, consistent with an embodiment of the present disclosure;

FIG. 8 illustrates an exemplary configuration of a winch assembly, consistent with an embodiment of the present disclosure;

FIG. 9A illustrates a top view of one example of hand positioning on an exemplary cardiac device, consistent with an embodiment of the present disclosure;

FIG. 9B illustrates a bottom view of the example of hand positioning on an exemplary cardiac device in FIG. 9A, consistent with an embodiment of the present disclosure;

FIG. 9C illustrates a top view of another example of hand positioning on an exemplary cardiac device, consistent with an embodiment of the present disclosure;

FIG. 9D illustrates a bottom view of the example of hand positioning on an exemplary cardiac device in FIG. 9C, consistent with an embodiment of the present disclosure;

FIG. 10A illustrates a snare actuator configuration, consistent with an embodiment of the present disclosure; and

FIG. 10B illustrates another snare actuator configuration, consistent with an embodiment of the present disclosure.

FIG. 11A illustrates an examples of a first configuration of the inner catheter in relation to the outer catheter.

FIG. 11B illustrates an example of a second configuration of the inner catheter in relation to the outer catheter.

### DETAILED DESCRIPTION OF EMBODIMENTS

The present disclosure relates to methods and devices for providing a cardiac device comprising a set of multiple nested catheters connected to a single handle.

Some embodiments include a set of at least two nested catheters including at least an outer catheter and an inner catheter. The term “catheter” refers generally to any element that is capable of extending or being inserted into an opening of a cavity, such as a chamber of a heart in the body. For example, a catheter may include guide catheters, balloon catheters such as PTA and PTCA catheters for angioplasty, catheters for prostate therapy, TTS endoscopic catheters for gastrointestinal use, single operator exchange or rapid exchange (SOE or RX) catheters, over-the-wire (OTW) catheters, fixed wire catheters, medical device delivery catheters including stent delivery devices in both the self-expanding and balloon expandable varieties, catheters for delivery of vena cava filters, catheters for delivery of percutaneous patent foramen ovale (PFO) closure devices, therapeutic substance delivery devices, thrombectomy devices, endoscopic devices, angiographic catheters, neuro catheters, dilatation catheters, urinary tract catheters, gastrointestinal catheter devices, heat transfer catheters including thermal catheters and cooling, intravascular ultrasound systems, electrophysiology devices, and any other device capable of insertion into a cavity. A set of two nested catheters including an outer catheter and an inner catheter may include an inner catheter placed inside an outer catheter. In some examples, the inner catheter may be placed entirely inside the outer catheter. In other examples, the outer catheter may be placed partially inside the outer catheter, such that only a portion of the inner catheter is positioned within the outer catheter.

Some embodiments include a single handle associated with the set of at least two nested catheters, the single handle including an outer shell and an inner catheter mount assembly configured to slide longitudinally within the outer shell. The term “handle” refers generally to any element by which an object is held, carried, or controlled, such as a shaft, stem, grip, or holder. A single handle associated with the set of at least two nested catheters refers to one handle being proximate to, corresponding to, matching, being attached to, being related to, or in any other way being connected to or with the at least two nested catheters. For example, the set of at least two nested catheters may be nested within the handle. As another example, the handle may be removably attached to the set of at least two nested catheters. An outer shell refers to any element that contains or encloses all or part of the handle. As an example, the outer shell may be an exterior casing of the handle. As another example, the outer shell may be a portion of the outside wall of the handle. An inner catheter mount assembly refers to a body, cluster, collection, group, or any other fitting together of parts into a complete machine, structure, or unit. For example, the inner catheter mount assembly may include a plurality of mechanical structures, such as gears, pins, or actuators associated with the inner catheter, within the outer shell of the handle.

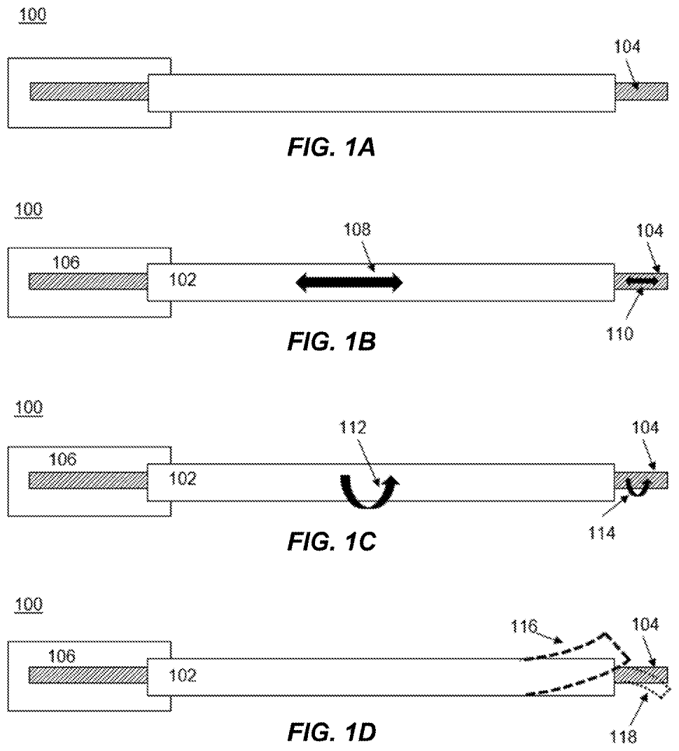

FIG. 1A illustrates an exemplary cardiac device 100, consistent with an embodiment of the present disclosure. Cardiac device 100 may include a set of two nested catheters 102 and 104, including an outer catheter 102 and an inner catheter 104. Cardiac device 100 may also include a handle 106 that is associated with the set of at least two nested catheters 102 and 104.

In some embodiments, the outer catheter is associated with the outer shell of the handle, such that translation of the outer shell is configured to translate the outer catheter and the inner catheter together, and rotation of the outer shell is configured to rotate the outer catheter and the inner catheter together. Translation refers to a movement of an object or a point in any direction, such as by sliding, shifting, migrating, progressing, relocating, advancing, or in any other way moving. For example, in a coordinate system, a translation may refer to the movement of a point from one position on the x-axis to another point on the x-axis. As another example, the translation may be a movement of a point from one position on the x-axis to a position on the y-axis. In the context of the set of nested catheters, translation of the outer catheter and the inner catheter together may refer to any movement of the outer catheter and the inner catheter in a way that is related, associated, or in any other way connected. For example, when the outer catheter slides distally by 5 cm, the inner catheter may also slide distally by 5 cm. The translation of the outer catheter may be the same as or different from the translation of the inner catheter. For example, when the outer catheter slides distally by 5 cm, the inner catheter may slide by 2 cm. Rotation refers to pivoting, revolving, spinning, swiveling, twisting, circling, rolling, or any other action of moving around an axis or center. In the context of the set of nested catheters, rotation of the outer catheter and the inner catheter together may refer to any movement of the outer catheter and the inner catheter around an axis or center in a way that is related, associated, or in any other way connected. For example, when the outer catheter rotates clockwise by 90 degrees, the inner catheter may also rotate clockwise by 90 degrees. The rotation of the outer catheter may be the same as or different from the rotation of the inner catheter. For example, when the outer catheter rotates clockwise by 90 degrees, the inner catheter may rotate clockwise by 70 degrees.

FIG. 1B illustrates a translational movement of an exemplary cardiac device 100, consistent with an embodiment of the present disclosure. The outer catheter 102 may have a translation 108 and the inner catheter 104 may have a translation 110. Translation 108 and translation 110 may have the same magnitude or different magnitudes.

FIG. 1C illustrates a rotational movement of an exemplary cardiac device 100, consistent with an embodiment of the present disclosure. The outer catheter 102 may have a rotation 112 and the inner catheter 104 may have a rotation 114. Rotation 112 and rotation 114 may have the same magnitude or have different magnitudes. Rotation 112 and rotation 114 may be in the same direction (clockwise-clockwise) or in different directions (clockwise-counterclockwise).

In some embodiments, the inner catheter is associated with the inner catheter mount assembly, such that sliding of the inner catheter mount assembly within the outer shell of the handle is configured to induce translation of the inner catheter relative to the outer catheter. Translation of the inner catheter relative to the outer catheter refers to a movement of the inner catheter in any direction, such as by sliding, shifting, migrating, progressing, relocating, advancing, or in any other way moving as observed from or relative to the outer catheter as a point of reference. For example, translation of the inner catheter relative to the outer catheter may involve only the inner catheter sliding in a distal direction while the outer catheter remains static. As another example, translation of the inner catheter relative to the outer catheter may involve both the inner catheter and the outer catheter sliding in a distal direction, wherein the inner catheter slides more or less distally than the outer catheter.

In some embodiments, the inner catheter mount assembly includes a rotatable knob configured to be rotated from an exterior of the handle, such that rotation of the rotatable knob is configured to induce rotation of the inner catheter relative to the outer catheter. A rotatable knob refers to any structure, often rounded, that may be used to control an object and is capable of being rotated. Rotation of the inner catheter relative to the outer catheter refers to the inner catheter pivoting, revolving, spinning, swiveling, twisting, circling, rolling, or any other action of moving around an axis or center, as observed from or relative to the outer catheter as a point of reference. For example, rotation of the inner catheter relative to the outer catheter may involve only the inner catheter turning in a clockwise direction while the outer catheter remains static. As another example, rotation of the inner catheter relative to the outer catheter may involve both the inner catheter and the outer catheter rotating in a clockwise direction, wherein the inner catheter rotates by a larger degree than the outer catheter.

In some embodiments, the outer shell includes a longitudinal slit, and the deflection actuator is configured to protrude through the longitudinal slit. A longitudinal slit refers to any long, narrow cut or opening in the outer shell capable of accommodating at least a portion of the deflection actuator. In some embodiments, the inner catheter mount assembly is configured to slide forwards and backwards along the longitudinal slit in the outer shell.

In some embodiments, the inner catheter mount assembly is associated with a first gear having a first plurality of teeth. A gear refers to a wheel with teeth configured to engage with the teeth of another wheel or linear gear to transmit rotational motion, optionally changing the speed and/or direction of transmitted rotational motion. A plurality of teeth refers to two or more of a projecting part, such as a spike, on a gear. In some embodiments, an inner surface of the rotatable knob includes a second plurality of teeth configured to engage the first plurality of teeth. In some embodiments, the inner catheter is associated with a second gear having a third plurality of teeth and the first plurality of teeth is configured to engage the third plurality of teeth. In some embodiments, the outer shell includes a longitudinal slit, and the first gear is configured to lie within the longitudinal slit. A longitudinal slit refers to any long, narrow cut or opening in the outer shell capable of accommodating at least a portion of the first gear, such as the first plurality of teeth.

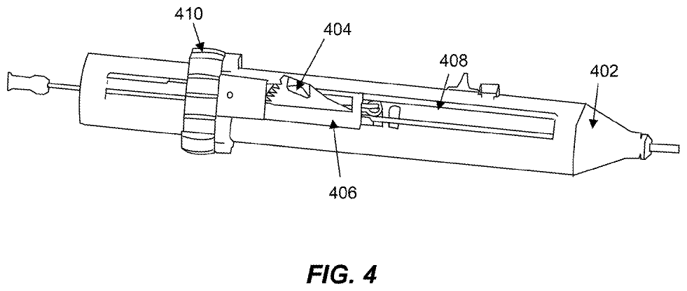

FIG. 4 illustrates an exemplary rotatable knob 410, consistent with an embodiment of the present disclosure. The device may include an inner catheter mount assembly 406, a longitudinal slit 408, and a handle outer shell 402. The inner catheter mount assembly 406 is configured to slide in longitudinal slit 408 in outer shell 402. The device may include an actuator 404 for inner catheter deflection that protrudes through longitudinal slit 408 in outer shell 402. The device may include a knob 410 for actuation of both translation and rotation of the inner catheter mount.

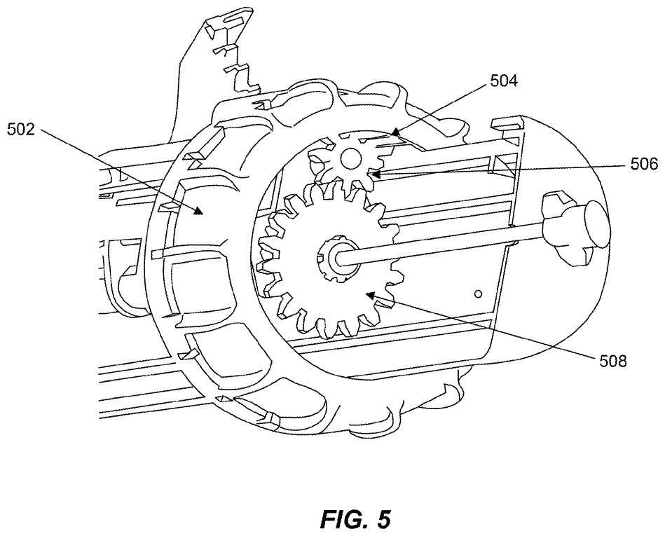

FIG. 5 illustrates the components of an exemplary rotatable knob 502, consistent with an embodiment of the present disclosure. Rotatable knob 502 may include teeth 504 on an inner surface of the knob 502. The device also includes a gear 508 attached to the inner catheter mount. The device further includes a gear 506 mounted on the inner catheter mount assembly and which lies within the longitudinal slit in the outer shell.

In some embodiments, the inner catheter mount assembly includes a deflection actuator configured to be actuated from an exterior of the handle, such that actuation of the deflection actuator is configured to induce deflection of a portion of the inner catheter that extends beyond a distal end of the outer catheter. A deflection actuator refers to any component or device configured to convert one or a combination of energy or signals to produce a motion of any object or point. A deflection actuator may be a mechanical actuator, such as a button. A deflection actuator may also be an electrical actuator, such as a piezoelectric strip. A deflection actuator may also be a combination of a mechanical actuator and an electrical actuator. Actuation refers to the resulting motion caused by an interaction with the deflection actuator. Actuation from an exterior of the handle refers to manipulation of any portion of the deflection actuator from outside the handle. As such, the entirety of the manipulation of the deflection actuator that causes the resulting motion may be from outside the handle. Alternatively, only a portion of the manipulation of the deflection actuator that causes the resulting motion may be from outside the handle.

FIG. 1D illustrates a deflection of an exemplary cardiac device 100, consistent with an embodiment of the present disclosure. The outer catheter 102 may deflect and assume a deflected condition or shape represented by deflection 116 and the inner catheter 104 may deflect and assume a deflected condition or shape represented by deflection 118. Deflection 116 and deflection 118 may have the same magnitude or may have different magnitudes. Deflection 116 and deflection 118 may be any of a bending or movement of the distal end of the outer catheter 102 or inner catheter 104, respectively, in any direction.

In some embodiments, the deflection actuator includes one of: a slider, lever, or button. A slider refers to any element, such as a protrusion of the inner catheter mount assembly, configured to be moved linearly along an axis to control the movement of a connected part or object. A lever refers to any element, such as a rigid bar, configured to move a part or object attached to or connected to the lever when pressure is applied to a location on the lever. A button refers to any element, such as a circular protrusion of the inner catheter mount assembly, configured to affect a movement when pressure is applied to a surface of the element.

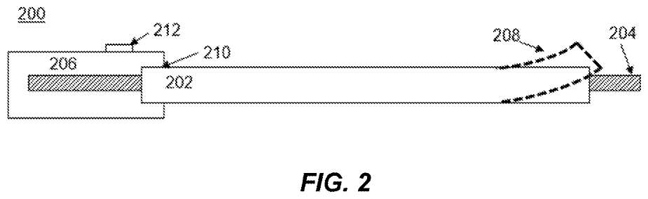

FIG. 2 illustrates an exemplary deflection actuator 212 of a cardiac device 200, consistent with an embodiment of the present disclosure. Cardiac device 200 may include a set of two nested catheters 202 and 204 including an outer catheter 202 and an inner catheter 204. Cardiac device 200 may also include a handle 206 that is associated with the set of two nested catheters 202 and 204. In the example in FIG. 2, deflection actuator 212 is a button that a user may press in order to cause deflection 208 of the outer catheter 202.

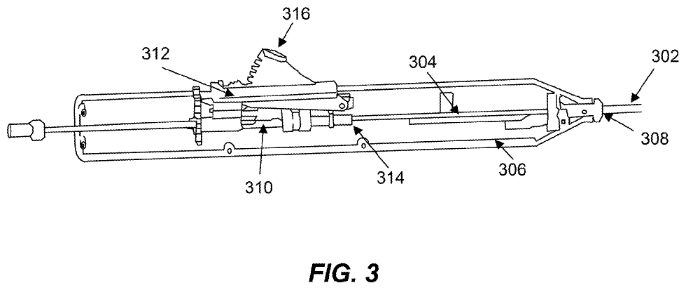

FIG. 3 illustrates the components of an exemplary deflection actuator 316 of a cardiac device, consistent with an embodiment of the present disclosure. The cardiac device may include an outer catheter 302, an inner catheter 304, and an outer shell 306 of the handle. In this example, the cardiac device includes an association 308 between the outer catheter 302 and the outer shell 306 of the handle that causes them to translate and rotate together, such as the outer catheter 302 being adhered to the outer shell 306 of the handle. The cardiac device of FIG. 3 also includes an inner catheter mount 310 configured to translate and rotate relative to the outer shell 306 of the handle. The cardiac device of FIG. 3 further includes an inner catheter mount assembly 312 which is configured to move within the outer shell 306 of the handle. The cardiac device also includes an association 314 between the inner catheter 304 and the inner catheter mount assembly 312 that causes them to translate and rotate together.

In some embodiments, a range of translational motion of the inner catheter relative to the outer catheter includes the inner catheter being positioned such that its distal end is distal to the distal end of the outer catheter. A range of translational motion refers to an amount of movement between upper and lower limits of the movement of the inner catheter in any direction, such as by sliding, shifting, migrating, progressing, relocating, advancing, or moving in any other way as observed from or relative to the outer catheter as a point of reference. For example, a range of translational motion of the inner catheter relative to the outer catheter may refer to the distal tip of the inner catheter being able to slide distally away from the distal tip of the outer catheter up to 5 cm, 10 cm, 15 cm, or any other distance.

In some embodiments, a total range of translation of the inner catheter relative to the outer catheter is greater than one third of an overall length of the handle. A total range of translation of the inner catheter relative to the outer catheter refers to the maximum amount of movement between positions of the inner catheter as observed from or relative to the outer catheter as a point of reference. It may be desirable to have the total range of translation of the inner catheter relative to the outer catheter be greater than one third of an overall length of the handle so that the inner catheter can travel further into a cavity of the body, such as a chamber of the heart. It is contemplated that in some embodiments, the total range may be one-fourth, half, three-fourths, or any other ratio relative to the overall length of the handle.

Some embodiments include a pull wire associated with the inner catheter mount assembly and connected to the inner catheter, the pull wire, upon manipulation, being configured to induce deflection of the inner catheter. A pull wire extends through a lumen of the inner catheter and a distal end of the pull wire may be secured at or near a distal end of the inner catheter.

Some embodiments include a pull ring associated with the pull wire, wherein the pull ring is configured to rotate with the inner catheter when the inner catheter is rotated. The pull ring may be associated with the pull wire in any way, such as anchored by welding the pull wire to a pull ring, wrapping the pull wire around a portion of the pull ring, and or using a glue or adhesive to connect the pull wire to the pull ring.

Some embodiments include a carriage associated with the inner catheter mount assembly, wherein the carriage is configured to remain unrotated relative to the outer shell when the inner catheter is rotated. A carriage refers to any movable part associated with the inner mount assembly. In some embodiments, the carriage is configured to push the pull ring back and forth when it moves back and forth to induce a deflection of the inner catheter.

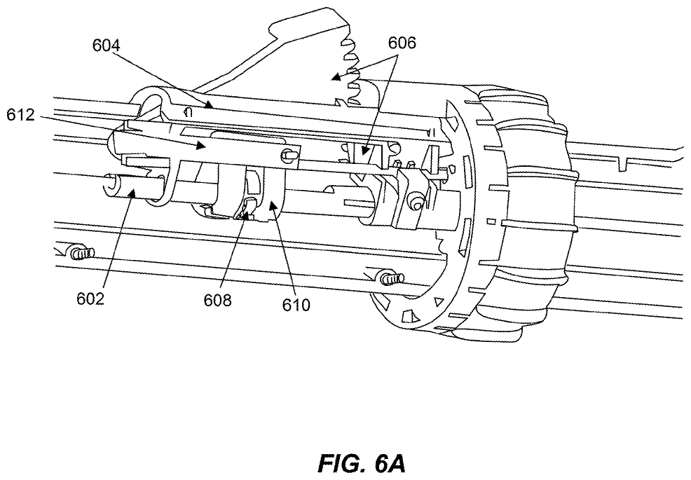

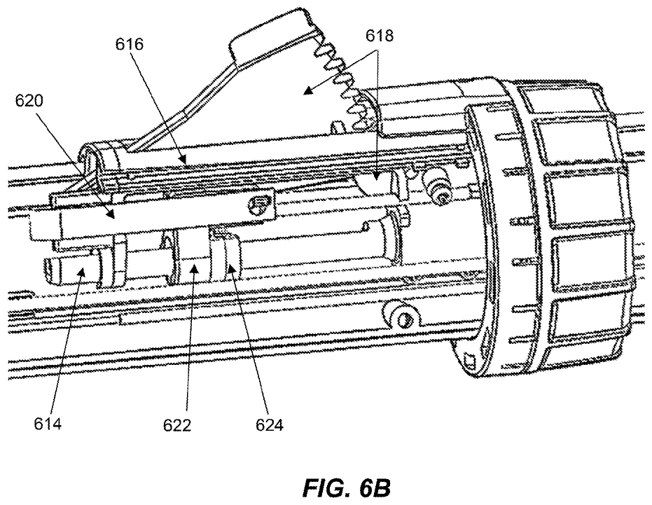

FIG. 6A illustrates the components of an exemplary inner catheter mount assembly 604, consistent with an embodiment of the present disclosure. The device includes an inner catheter mount assembly 604 and a carriage 610 associated with the inner catheter mount assembly 604. The carriage 610 does not rotate with the inner catheter mount 602, while the pull ring 608 does rotate with the inner catheter mount 602. The spring 612 moves carriage 610 forwards, while the deflection actuation mechanism 606 is configured to pull carriage 610 backwards.

In some embodiments, the carriage is configured to push the pull ring back when it moves back, to induce a deflection of the inner catheter, but not to push the pull ring forward when it moves forward. In some embodiments of the invention the carriage is pulled back by a deflection actuation mechanism, and is allowed to move forward by the passive straightening of the catheter. Optionally a spring is used to move the carriage forward if the passive straightening of the catheter is insufficient to overcome the friction in the mechanism.

Optionally, the carriage is configured to push the pull ring back when it is pulled back by a deflection actuation mechanism, but not to push the pull ring forward when it moves forward due to the force in the spring, rather allowing the pull ring to move forward due to the passive straightening of the catheter without needing to overcome the friction in the mechanism which has been overcome by the spring.

FIG. 6B illustrates an exemplary inner carriage configuration, consistent with an embodiment of the present disclosure. As shown in FIG. 6B, the device includes an inner catheter mount 614 and an inner catheter mount assembly 616. The device also includes a deflection actuation mechanism 618 configured to pull carriage 622 backwards. The device further includes a spring 620 configured to move carriage 622 forwards. The pull ring 624 rotates with the inner catheter mount 614. The carriage 622 does not rotate with the inner catheter mount 614. The carriage 622 pushes pull ring 624 back but does not push it forwards.

Some embodiments include a winch and a cable connected to the carriage. A winch refers to any mechanical device configured to pull in (wind up) or let out (wind out) or otherwise adjust the tension of a cable. Examples of a winch include winches that are electrical, mechanical drum-style, mechanical capstan-style, hydraulic, mechanical hand-operated, mechanical portable, or hybrid. A cable refers to any wire or rope by which force is exerted to control or operate a mechanism, such as the carriage.

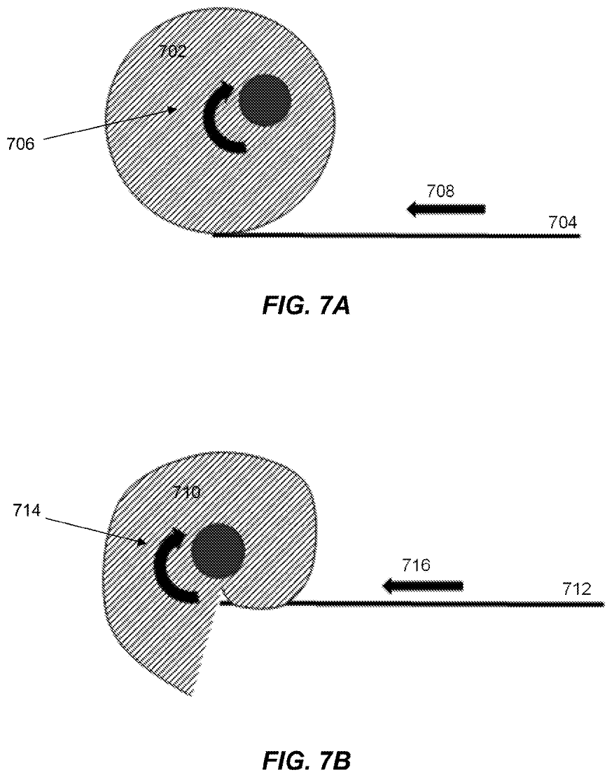

In some embodiments, the winch is configured to be angularly rotated in order to induce a linear movement of the carriage. An example of a winch configured to be angularly rotated in order to induce a linear movement of the carriage is an excentric winch, which has an axis that is not placed centrally and is configured to provide a non-linear relationship between the angular rotation of the winch and the linear motion of the object being pulled by the winch. In some embodiments, the winch is excentric causing a non-linear relationship between the angular rotation of the winch and the linear movement of the carriage. FIG. 7A illustrates one example of a winch 702, consistent with an embodiment of the present disclosure. In FIG. 7A, winch 702 is an excentric winch configured to provide a non-linear relationship between the angular rotation 706 of the winch 702 and the linear motion 708 of the object 704 being pulled by the winch 702.

In some embodiments, the winch has a portion with a non-circular shape of variable radius causing a non-linear relationship between the angular rotation of the winch and the linear movement of the carriage. FIG. 7B illustrates an example of a winch 710, consistent with an embodiment of the present disclosure. In FIG. 7B, winch 710 is a variable radius winch configured to provide a non-linear relationship between the angular rotation 714 of the winch 710 and the linear motion 716 of the object 712 being pulled by the winch 710.

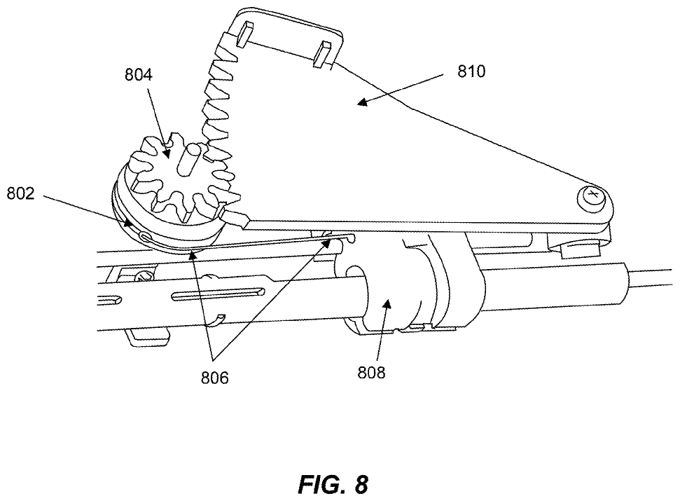

FIG. 8 illustrates an exemplary configuration of a winch assembly, consistent with an embodiment of the present disclosure. In FIG. 8, excentric winch 802 includes a gear 804. The device includes a lever 810, carriage 808, and cable 806. Cable 806 is configured to be pulled by winch 802, which pulls the carriage 808 backwards.

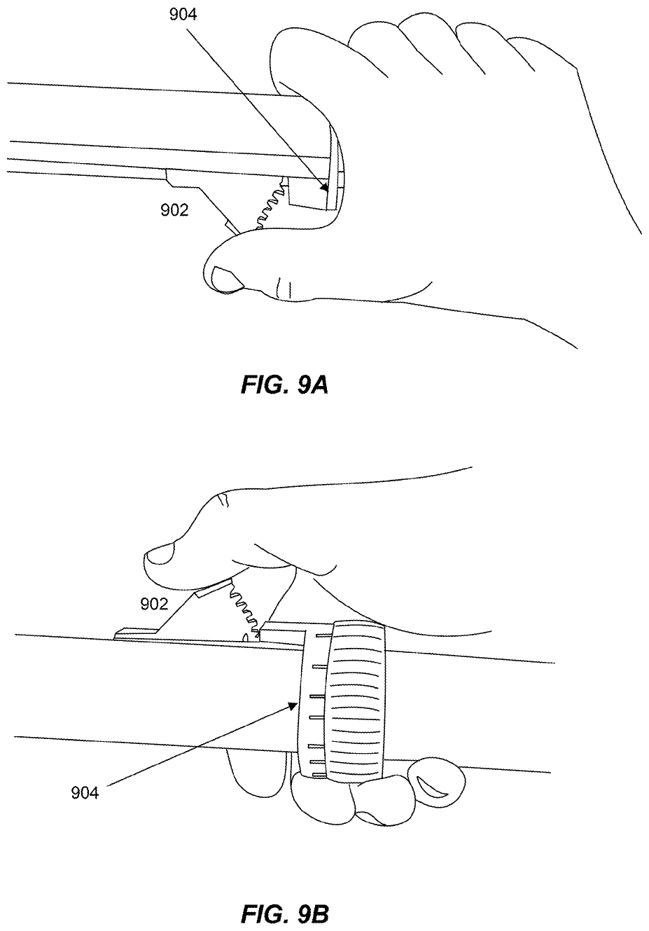

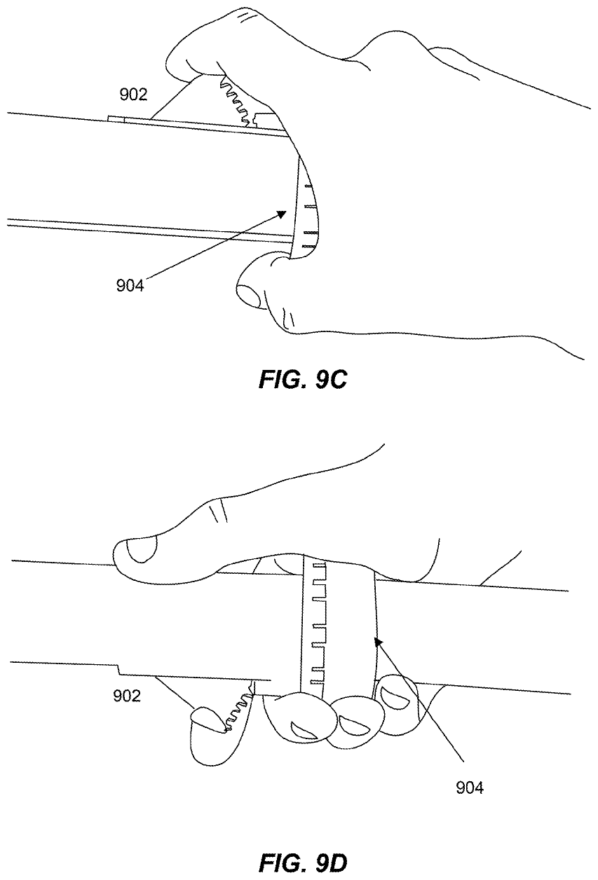

FIG. 9A illustrates a top view of one example of hand positioning on an exemplary cardiac device, consistent with an embodiment of the present disclosure. FIG. 9B illustrates a bottom view of the example of hand positioning on an exemplary cardiac device in FIG. 9A, consistent with an embodiment of the present disclosure. In this example, a user's thumb is used to control lever 902 and a user's palm and fingers are used to control rotatable knob 904. FIG. 9C illustrates a top view of another example of hand positioning on an exemplary cardiac device, consistent with an embodiment of the present disclosure. FIG. 9D illustrates a bottom view of the example of hand positioning on an exemplary cardiac device in FIG. 9C, consistent with an embodiment of the present disclosure. In this example, a user's thumb and palm are used to control rotatable knob 904 and a user's index finger is used to control lever 902. As shown in FIGS. 9A-9D, some disclosed embodiments of the cardiac device enable manipulation of both the rotatable knob and the deflection actuator simultaneously.

Some embodiments include a snare connected to a snare wire configured to be located within a lumen of one of the outer catheter or the inner catheter. The term “snare” may generally refer to any mechanism that is capable of being mounted on a wire or a thin tube (for example, at a distal end thereof) and that can be used to capture and grasp an object, such as a guidewire.

In some embodiments, the snare wire is connected to a snare actuator located in the handle, such that actuation of the snare actuator is configured to move the snare from a concealed position within the lumen to an exposed position outside the lumen. A snare actuator refers to any component or device configured to convert one or a combination of energy or signals to produce a motion of the snare. A concealed position within the lumen refers to any position of the snare that is partially or wholly covered, veiled, enclosed, obscured, or otherwise positioned inside the lumen. An exposed position outside the lumen refers to any position of the snare that is partially or wholly uncovered by, unsheathed from, or otherwise positioned external to the lumen. In some embodiments, the snare actuator includes a snare locking mechanism configured to lock the snare in the concealed position within the lumen. In some embodiments, the snare locking mechanism is configured to release the snare from the concealed position in response to a user action.

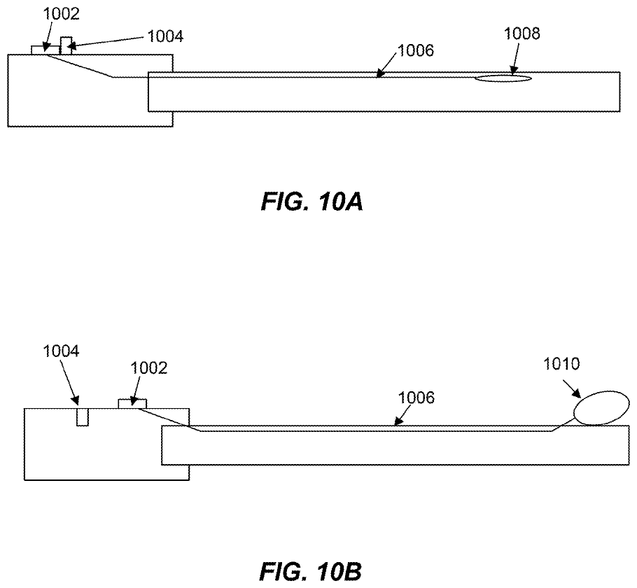

FIG. 10A illustrates a snare actuator configuration associated with a concealed snare, consistent with an embodiment of the present disclosure. FIG. 10B illustrates another snare actuator configuration associated with an exposed snare, consistent with an embodiment of the present disclosure. The device includes a snare actuator 1002, snare actuator lock mechanism 1004, snare wire 1006, and snare in a concealed position 1008. The snare actuator lock mechanism 1004 in this example may be a button that once depressed, allows snare actuator 1002 to be slid by a user distally, allowing the snare in a concealed position 1008 to be moved distally to an exposed position 1010.

In some embodiments, the inner catheter includes a distal tip with an outer diameter larger than the inner diameter of the outer catheter. In some embodiments, the outer diameter of the distal tip of the inner catheter is between 3 mm and 4 mm. In some embodiments, the outer diameter of the distal tip of the inner catheter is similar to, optionally within 1 mm or 0.5 mm of, the outer diameter of the outer catheter. In some embodiments, the distal tip of the inner catheter is rounded and smooth in order to be atraumatic. In some embodiments, the transition from the large diameter distal tip to the small diameter inner catheter shaft is a smooth gradual transition in order that the catheter should not get stuck on tissue when manipulated within the ventricle of a heart.

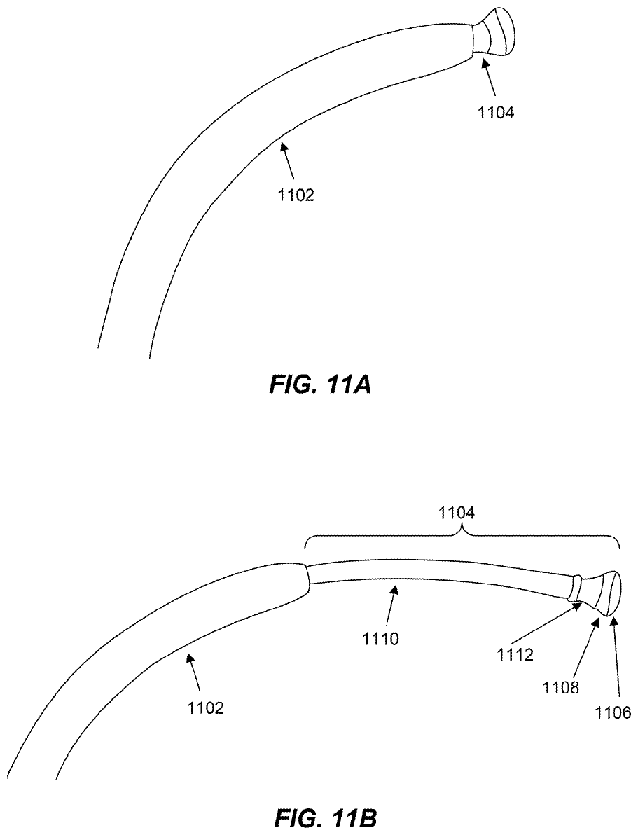

FIG. 11A illustrates an example of a first configuration of the inner catheter in relation to the outer catheter. As shown in FIG. 11A, the inner catheter 1104 is nested within outer catheter 1102 in a first configuration. FIG. 11B illustrates an example of a second configuration of the inner catheter in relation to the outer catheter. As shown in FIG. 11B, the inner catheter 1104 is exposed from the outer catheter 1102 in a second configuration. This second configuration may be achieved by translating the inner catheter 1104 out of the outer catheter 1102 using a handle, such as in the embodiments disclosed herein. Referring to FIG. 11B, inner catheter 1104 includes a rounded tip 1106. Additionally, the transition 1112 from the large diameter distal tip 1108 to the small diameter inner catheter shaft 1110 is a smooth gradual transition in order that the catheter does not get stuck on tissue when manipulated within the ventricle of a heart.

While the present disclosure is described herein with reference to illustrative embodiments of a cardiac device used for particular applications, such as for navigation through chambers of a heart for cardiac repair or inspection, it should be understood that the embodiments described herein are not limited thereto. Those having ordinary skill in the art and access to the teachings provided herein will recognize additional modifications, applications, embodiments, and substitution of equivalents that all fall within the scope of the disclosed embodiments. Accordingly, the disclosed embodiments are not to be considered as limited by the foregoing or following descriptions.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description.

1. A cardiac device, comprising:
a set of at least two nested catheters including at least an outer catheter and an inner catheter; and
a single handle associated with the set of at least two nested catheters, the single handle including an outer shell and an inner catheter mount assembly configured to slide longitudinally within the outer shell, wherein:
the outer catheter is associated with the outer shell of the handle, such that translation of the outer shell is configured to translate the outer catheter and the inner catheter together, and rotation of the outer shell is configured to rotate the outer catheter and the inner catheter together;
the inner catheter is associated with the inner catheter mount assembly, such that sliding of the inner catheter mount assembly within the outer shell of the handle is configured to induce translation of the inner catheter relative to the outer catheter;
the inner catheter mount assembly includes a rotatable knob configured to be rotated from an exterior of the handle, such that rotation of the rotatable knob is configured to induce rotation of the inner catheter relative to the outer catheter;
the inner catheter mount assembly includes a deflection actuator configured to be actuated from an exterior of the handle, such that actuation of the deflection actuator is configured to induce deflection of a portion of the inner catheter that extends beyond a distal end of the outer catheter; and
a range of translational motion of the inner catheter relative to the outer catheter includes:
the inner catheter being positioned such that its distal end is distal to the distal end of the outer catheter; and
a total range of translation of the inner catheter relative to the outer catheter is greater than one third of an overall length of the handle.

2. The cardiac device of claim 1, wherein the outer shell includes a longitudinal slit, and the deflection actuator is configured to protrude through the longitudinal slit.

3. The cardiac device of claim 2, wherein the inner catheter mount assembly is configured to slide forwards and backwards along the longitudinal slit in the outer shell.

4. The cardiac device of claim 1, wherein the inner catheter mount assembly is associated with a first gear having a first plurality of teeth.

5. The cardiac device of claim 4, wherein an inner surface of the rotatable knob includes a second plurality of teeth configured to engage the first plurality of teeth.

6. The cardiac device of claim 5, wherein the inner catheter is associated with a second gear having a third plurality of teeth and wherein the first plurality of teeth is configured to engage the third plurality of teeth.

7. The cardiac device of claim 4, wherein the outer shell includes a longitudinal slit, and the first gear is configured to lie within the longitudinal slit.

8. The cardiac device of claim 1, wherein the deflection actuator includes one of: a slider, lever, or button.

9. The cardiac device of claim 1, further comprising a pull wire associated with the inner catheter mount assembly and connected to the inner catheter, the pull wire, upon manipulation, being configured to induce deflection of the inner catheter.

10. The cardiac device of claim 9, further comprising a pull ring associated with the pull wire, wherein the pull ring is configured to rotate with the inner catheter when the inner catheter is rotated.

11. The cardiac device of claim 10, further comprising a carriage associated with the inner catheter mount assembly, wherein the carriage is configured to remain unrotated relative to the outer shell when the inner catheter is rotated.

12. The cardiac device of claim 11, wherein the carriage is configured to push the pull ring back and forth when it moves back and forth to induce a deflection of the inner catheter.

13. The cardiac device of claim 11, wherein the carriage is configured to push the pull ring back when it moves back, to induce a deflection of the inner catheter, but not to push the pull ring forward when it moves forward.

14. The cardiac device of claim 11, further comprising a winch and a cable connected to the carriage.

15. The cardiac device of claim 14, wherein the winch is configured to be angularly rotated in order to induce a linear movement of the carriage.

16. The cardiac device of claim 14, wherein the winch has a portion with a non-circular shape of variable radius causing a non-linear relationship between the angular rotation of the winch and the linear movement of the carriage.

17. The cardiac device of claim 14, wherein the winch is excentric causing a non-linear relationship between the angular rotation of the winch and the linear movement of the carriage.

18. The cardiac device of claim 1, further comprising a snare connected to a snare wire configured to be located within a lumen of one of the outer catheter or the inner catheter.

19. The cardiac device of claim 18, wherein the snare wire is connected to a snare actuator located in the handle, such that actuation of the snare actuator is configured to move the snare from a concealed position within the lumen to an exposed position outside the lumen.

20. The cardiac device of claim 19, wherein the snare actuator includes a snare locking mechanism configured to lock the snare in the concealed position within the lumen.
